(19) United States
(12) Patent Application Publication
(10) Pub. No.: US 2004/0081692 A1
(43) Pub. Date: Apr. 29, 2004

(54) Oral ndds design using modeling based on fractional release times

(76) Inventor: Vinay Sharma, Long Valley, NJ (US)

Correspondence Address: Louis E. Marn, 9 Edgewater Drive, Palm Coast, FL, 32164, US

(21) Appl. No.: 10/386,074

(22) Filed: Mar. 11, 2003

Related U.S. Application Data
(60) Provisional application No. 60/363,392, filed on Mar. 11, 2002.

(51) Int. Cl. A61K 9/22; G06F 19/00; G01N 33/48; G01N 33/50

(52) U.S. Cl. 424/468; 702/19

(57) ABSTRACT

There is disclosed a method for correlating in-vitro to in-vivo drug release for small molecules demonstrating a non-linear relationship for point-to-point analysis without emphasizing time scales with a convolution-based predictive model for designing modified release products eliminating parameters of in-vitro dissolution, such as pH and hydrodynamics.

### RELATED APPLICATION

This application claims the benefit of U.S. Provisional application No. 60/363,392, filed Mar. 11, 2002.

### BACKGROUND OF THE INVENTION

1. Field of the Invention

A “Level A” IVIVC procedure is developed for small molecules to correlate in-vitro to in-vivo drug release. This approach demonstrates a non-linear relationship for point-to-point analysis without emphasizing time scales and a convolution-based predictive model for designing modified release products.

2. Description of the Prior Art

Sharma and Khan have published fractional release time approach which relies on comparison of times for fractional release of drug from test and innovator for establishing bioequivalence. “FRT in Oral NDDS Design”, Express Pharma Pulse, Nov. 22, 2001.

Henry J. Malinowski presented Regulatory Applications of IVIVC in ER Drug Development In: Proceedings of Controlled Release Society Workshop on In-Vitro/In-Vivo co-relation applicable to extended release formulations held at Paris France, July 8-9, 2000. Sponsored by MPS and APV. The speaker quoted the example of Enteric Coated Mulitple Unit Dosage Forms requiring special techniques to establish IVIVC.

Peter Veng-Pedersen presented in the above Workshop, System Analysis: Introduction to Convolution and Deconvolution as Applied to In-Vitro/In-Vivo Correlations He discussed several possibilities such as absorption rate is assumed equal to the in vitro release rate, truncation of absorption profile, or non-linear linking corresponding to a saturable, pre-systemic elimination.

Jennifer Dressman stresses the need to develop physiologically representative conditions in the beaker in order to exhibit the desired release profiles in vivo.—Jennifer B. Dressman, JW Goethe University, Germany, Pharm. Res. 15:698 (1998).: Evaluation of various dissolution media for predicting in vivo performance of class I and class II drugs.

GastroPlus™ is a simulation software designed to predict the percent of a drug taken orally. Here, the percent absorbed is defined as the percent of the dose that reaches the compartment designated as “portal vein”. The IVIVC module is included in this software.

Adrian Dunne, Thomas O'Hara, and John Devane have described non-linear models for the relationship between the fraction of drug dose dissolved (absorbed) in vivo and that dissolved in vitro. Level A in-Vivo-in Vitro Correlation: Nonlinear Models and Statistical Methodology. Journal of Pharmaceutical Sciences, Vol 86, No. 11, 1245-1249 (1997). These authors attribute the lack of meaningful correlations between in vitro dissolution rates and rate and extent of bioavailability (as determined by blood concentrations and/or urinary excretion of drug or metabolites) to the restrictive nature of the linear model currently employed.

The ability to predict in-vivo performance from in-vitro dissolution is invaluable. A ‘level A’ IVIVC is considered to be the most informative and is recommended by the FDA. Most commonly seen process for developing ‘level A’ IVIVC is to develop formulations with different release rates and estimate in-vivo times using deconvolution and relate in-vitro to in-vivo. A prerequisite for an acceptable IVIVC is that in-vitro dissolution methodology should be discriminating. Dissolution conditions may be altered to develop ‘level A’ correlation and/or time scaling may be used to establish IVIVC.

There are techniques available to predict in-vivo performance from in-vitro dissolution studies based upon the use of IVIVC. The predictions, however are as good as the IVIVC itself. The predictability of in-vivo performance (in accordance with the FDA guidelines) will validate the method used.

To arrive at a ‘Level A’ IVIVC, there has to be a one to one correspondence between the iv-vivo and in-vitro release rates. If the two release rates are spread over a different time span, suitable treatment must be used to establish correspondence. This can be accomplished by modifying the dissolution technique and/or by modifying time scale by using one of the following:

Change dissolution apparatus from USP II to III or IV.

Change media from water to phosphate buffers, an alternative dissolution media to simulate fasted and prandial conditions.

Change hydrodynamics.

Use suitable transformation to establish correspondence.

In the absence of acceptable dissolution conditions to match the time spans of in-vitro and in-vivo release data, scaling of the time is needed. The time scaling can be achieved by compression of either of the in-vitro or in-vivo time scales. Appropriate transformation function may be needed if the in-vitro drug release deviates from the in-vivo release due to physiological conditions, such as the case with delayed release, site-specific or insoluble drug containing dosage forms.

Empirically, it might be possible to design a discriminating dissolution procedure for fasted and fed studies as reported by Dressman, however, it is far from the ideal because physiological environment cannot be created in a beaker.

Our approach is based on IVIVC using fractional release times (FRT) and is validated by several case studies. Basically, the FRT approach relies on comparison of times for fractional release of drug from test and innovator for establishing bioequivalence.

Duration of absorption of an MR (modified release) design must be critically evaluated using deconvolution because the extent of absorption of the active drug is related to the passage of the dosage form through the GI tract. Literature indicates that entire drug quantity is not absorbed if it is not delivered at the site of absorption.

Limitations in Prior Art

“Majority of attempts at establishing a level A in vitro to in vivo correlation (IVIVC) are based on a linear model.” Refer to Level A in-Vivo-in Vitro Correlation: Nonlinear Models and Statistical Methodology. Journal of Pharmaceutical Sciences, Vol 86, No. 11, 1245-1249 (1997). The following situations

If the drug is not absorbed at the same rate and extent from various sites of the GI tract, the linear IVIVC approach will not permit meaningful correlations between in vitro dissolution rates and rate and extent of availability as determined by blood concentrations and/or urinary excretion of drug or metabolites. Brockmeier has published a new approach under “Comparison of in Vitro and In Vivo Dissolution for the Study of Colonic Drug Absorption”, In: Colonic Drug Absorption and Metabolism, Editor Peter R. Bieck, Publisher Marcel Dekker, New York, 1993, pp 109. This approach focuses on continuous (point by point) comparison of the in vivo (hypothetical in vivo dissolution profiles) with an in vitro dissolution profile. The concept is based on linear transformation of the time axes of either. If superimosable after linear transformation, they are called homophormic., in contrast with isomorphic which are superimposable without transformation. If the situation is not covered under the two categories, it has to be handled with some composite technique such as the one presented here.

The present state of science and technology warrants a non-linear approach for modeling because it addresses more complicated drug delivery systems such as systems.based on time controlled, rate controlled, or spatially controlled drug delivery systems. The approach must be such as to include conventional models of linearity as a special case.

The United States Pharmacopeia lists about half a dozen dissolution apparatuses using variations of hydrodynamics and pH in response to industry's need to provide acceptable IVIVC to the Food and Administration. Modern trends in product design require such discriminating dissolution procedures for establishing IVIVC that the purpose of design is questionable if the empiricism of media/hydrodynamics manipulation is not properly justified.

Advantages of the FRT-IVIVC Approach

The approach of the present invention allows a Level A IVIVC while reducing the stringency of time scales and discriminating dissolution procedures. Having accepted that physiology dictates the in vivo component of IVIVC and the in vitro component is controlled by the dissolution procedure, the FRT approach uses the comparison of the two to estimate target vitro rates. As much as the FRT/IVIVC procedure allows the estimation of target vitro rates, conversely the procedure will allow the development of programmable delivery sytems having highly complex kinetic profiles.

### OBJECTS OF THE INVENTION

An object of the present invention is to demonstrate a non-linear relationship for point-to-point analysis without emphasizing time scales.

Another object of the present invention is to develop a convolution-based predictive model for designing modified release products.

Still another object of the present invention is to provide desired therapeutic ranges for several oral new drug delivery systems.

Another object of the present invention is to develop programmable drug delivery systems.based on time controlled, rate controlled, or spatially controlled drug delivery systems.

### SUMMARY OF THE INVENTION

These and other objects of the present invention are achieved by a “Level A” IVIVC procedure that is developed for small molecules to correlate in-vitro to in-vivo drug release. This approach demonstrates a non-linear relationship for point-to-point analysis without emphasizing time scales and a convolution-based predictive model for designing modified release products, without referring to the stringent criticality of parameters of in-vitro dissolution such as pH and hydrodynamics.

### BRIEF DESCRIPTION OF THE DRAWINGS

FIG. 1 PULSATILE MACROPARTICULATE DRUG DELIVERY SYSTEM (Showing hypothetical cumulative in-vivo dissolution profiles)

FIG. 2 HYDROPHILIC MATRIX DRUG DELIVERY SYSTEMS (Polyexponential Fitting of Fractional Release Time Based IVIVC)

FIG. 3 HYDROPHILIC MATRIX DRUG DELIVERY SYSTEMS (Simulation of Claritin D—12 Hour.)

FIG. 4 HYDROPHILIC MATRIX DRUG DELIVERY SYSTEMS (Fractional Release Time Based IVIVC for Claritin D—24 Hour.)

FIG. 5 HYDROPHILIC MATRIX DRUG DELIVERY SYSTEMS (Predicted in-vivo cumulative amounts for 12 Hours based on FRT IVIVC for 24 Hours of FIG. 3.)

FIG. 6 HYDROPHILIC MATRIX DRUG DELIVERY SYSTEMS (Predicted Human response for 12 Hours based on convolution of in-vivo amounts in FIG. 4.)

FIG. 7 DELAYED RELEASE DRUG DELIVERY SYSTEM (Showing hypothetical cumulative in-vivo dissolution profiles)

FIG. 8 DELAYED RELEASE DRUG DELIVERY SYSTEM (Showing hypothetical cumulative in-vivo dissolution profiles)

FIG. 9 DIFFUSION CONTROLLED DRUG DELIVERY STSTEM. (Linear Fitting of Fractional Release Time Based IVIVC)

### DETAILED DESCRIPTION OF THE INVENTION

THEORY:

The fundamental objective of in vitro/in vivo correlation (IVIVC) is to relate in vitro drug release with the drug presentation rate in vivo. Deconvolution is a mathematical tool by which in vivo drug presentation may be estimated. Convolution permits prediction of an expected human response, given an invivo presentation rate. The combination of IVIVC and convolution permits the prediction of an in vivo response given an in vitro presentation rate.

The convolution integral,

$$c ( t ) = {\int }_{0}^{t} f ( τ ) {c}_{δ} ( t - τ ) ⅆ τ$$

is the fundamental expression of convolution, where c_{δ} is the UIR (Unit Impulse Response). It is used for any drug exhibiting the quality of linearity.

To be linear, the system must have two qualities,

1. Superposition

2. Time invariance.

The quality of superposition may be stated as follows:

Given that the input f(t) produces the response c(t), superposition holds if a×f(t) produces the response a×c(t). Time invariance holds if f(t) produces the same c(t) irrespective of when f(t) is initiated. The first step in the FRT approach for establishing IVIVC is to obtain in vivo release through the deconvolution of a plasma-time profile of the innovator product. The second step is to convert the rates so obtained into cumulative amounts. A similar cumulative information is generated on in-vitro rates of the innovator product, using suitable dissolution conditions. From the corresponding cumulative amounts of in-vitro and in-vivo data, fractional release times (FRTᵥᵢₜᵣₒ and FRTᵥᵢᵥₒ, respectively) are computed. A Cartesian coordinate plot is prepared by plotting FRTᵥᵢₜᵣₒ on the X-axis and FRTᵥᵢᵥₒ on the Y-axis. This plot serves for the IVIVC of the innovator product. This plot is fitted with suitable function representing the relationship. For a simple relationship, the function can be approximated to be a straight line, Y=mx+c. Under certain conditions, the relationship may not be that simple and functions like polynomial, polyexponential etc. can be used.

### EXAMPLES

The premise of the use of FRT in establishing IVIVC and the subsequent prediction of human response using convolution was validated severally through the treatment of technologies such as

### Example 1

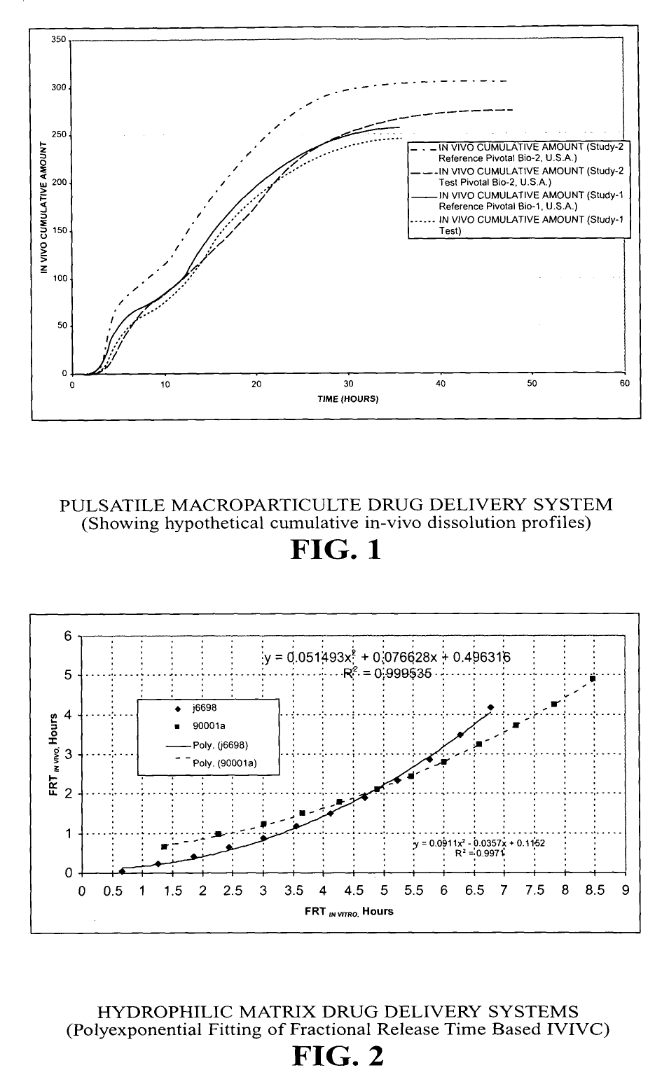

Pulsatile Macroparticulate Drug Delivery System (FIG. 1)

A time controlled, multi-pellet delivery system delivering water-soluble molecules and/or water dispersible nano-particles through a plasticized polymethacrylate membrane. Our technology utilizes the Wurster drug suspension layering and polymethacrylate membrane coating system to design multiple lag times and release rates for site-specific delivery. The term pulsatile is used to describe the rapid delivery of a dose of the drug (d₁, d₂, . . . dₙ) into the portal system over a specific time interval (Δₜ₁, Δₜ₂ . . . Δₜₙ) respectively preceded by lag times of (t_{lag1}, t_{lag2} . . . t_{lagn}). This regimen is equivalent to the rate of drug release from immediate release dosage forms. The shape of pulsatile rate of drug delivery is characterized by a gaussian distribution.

### Example 2

Hydrophilic Matrix Drug Delivery Systems

A. For Insoluble Drugs (FIG. 2)

Uses a highly swellable, water-soluble polymer and co-processed drug/cyclodextrin. There have been three U.S. patents filed for this technology. One of the patents represents a versatile process patent that allows controlled drug particle production, surface modification while maintaining particle crystallinity.

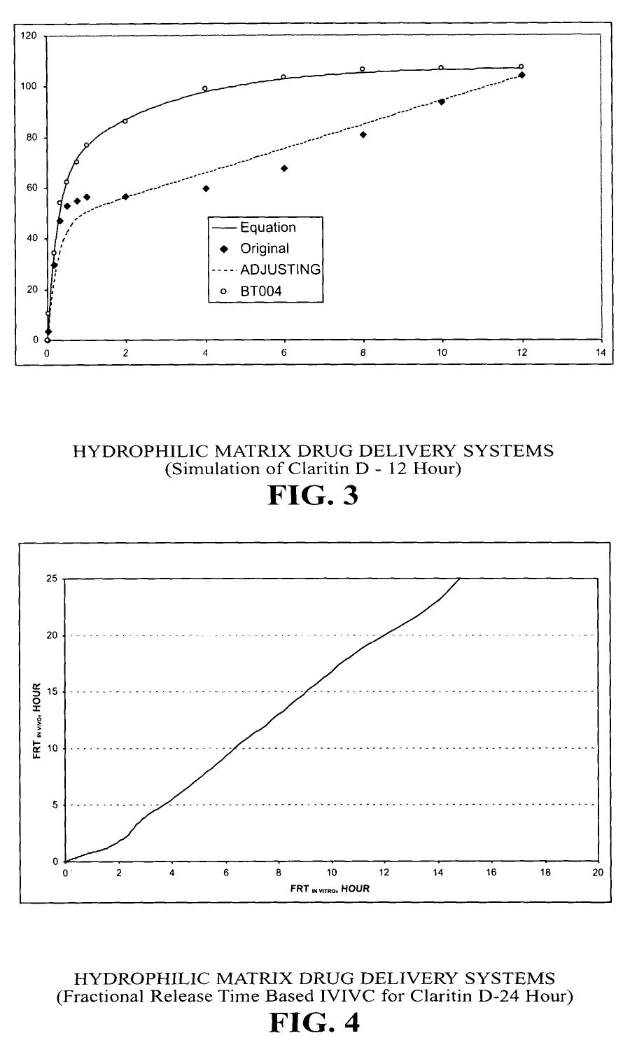

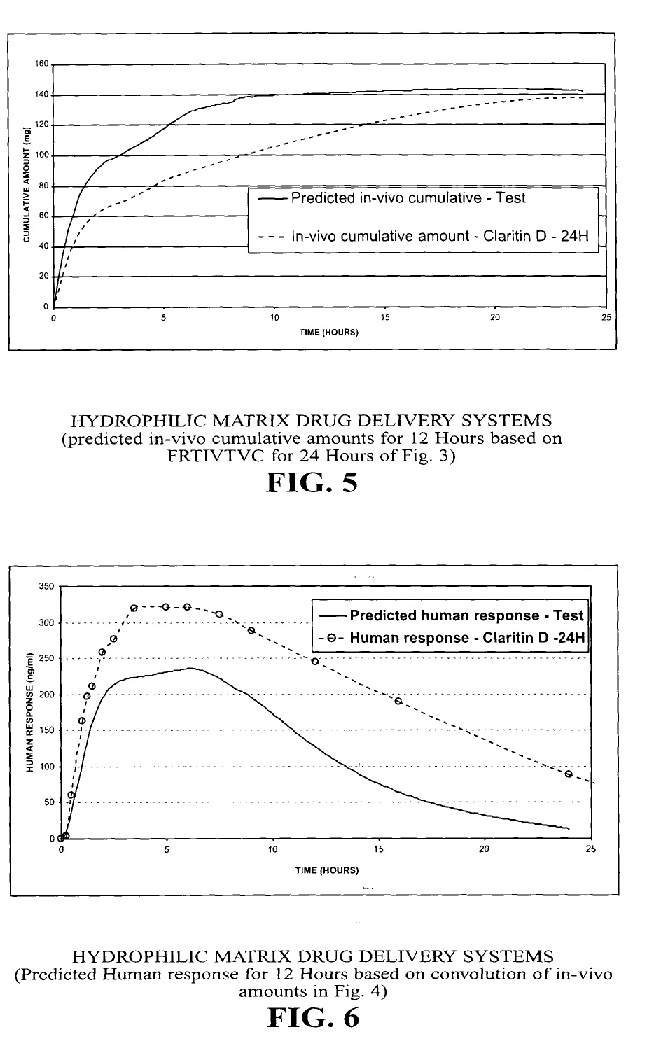

B. For Soluble Drugs—(FIGS. 3-6)

A biphasic release kinetics comprising a pulsatile, time-dependent component and a rate-controlled release component. was achieved by modeling, First, the human response from the 24-hr was deconvoluted to obtain in-vivo release rate. The second step is to convert the rates so obtained into cumulative amounts. From the corresponding cumulative amounts of the in-vitro and in-vivo release data for the 24-hr data, an IVIVC is generated based on fractional release times. The in-vitro data from the current invention of twice-daily dosing is used to compute the in-vivo release for the current invention. Then, using the technique of convolution, a human response is generated for the twice-daily dosing

### Example 3

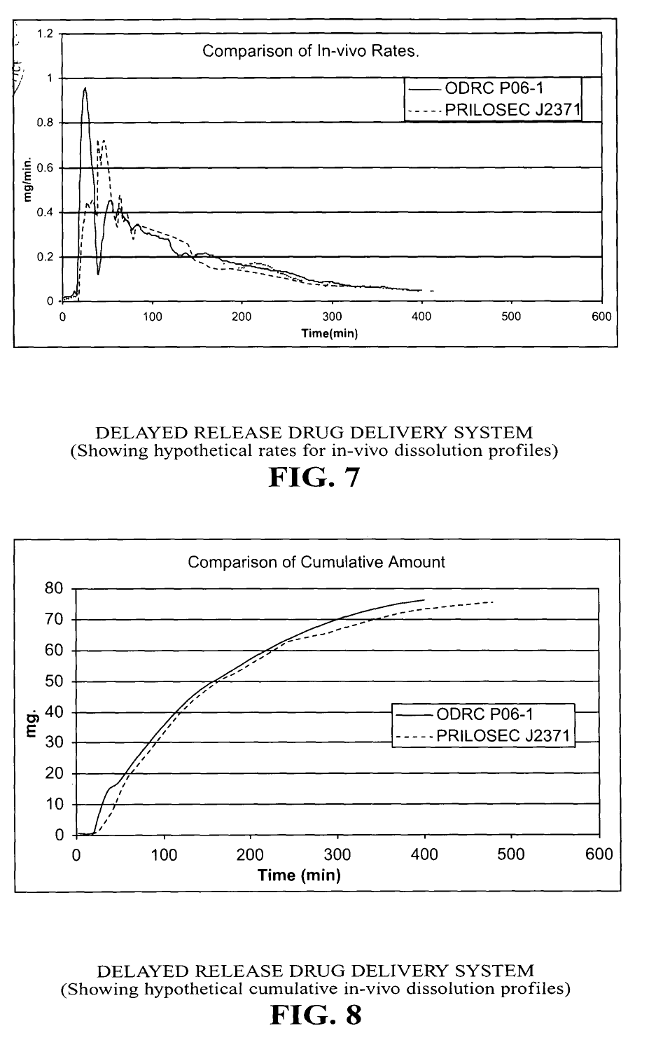

Delayed Release Drug Delivery System (FIGS. 7-8)

A multi-compartmental approach to alternate layering of microfluidized drug and a functional excipient, in a micro fluidized suspension for improved bioavailibility and stability performance. A final enteric coating or modified enteric coating is used to achieve gastro-resistance.

### Example 4

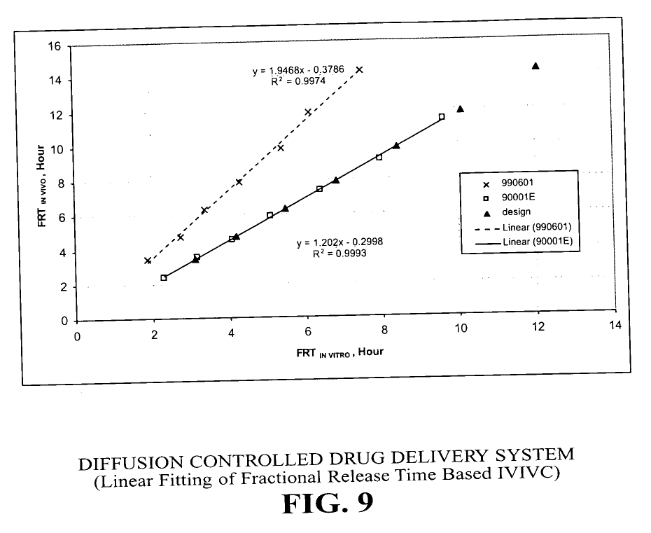

Diffusion Controlled Drug Delivery System (FIG. 9)

The product was developed by applying a plasticized water-insoluble membrane on dispersion layered pellets on non-pareils traded as NuPareils® or Celpheres®. The product was scaled up from GPCG-5 (7″ Wurster insert) to GPCG-120 (18″ Wurster insert). Appropriate SEM pictures were taken to relate membrane thickness to drug release as a function of scale-up. A human response was generated and deconvoluated using numerical deconvolution. Fractional response times were computed (FRTᵥᵢₜᵣₒ and FRTᵥᵢᵥₒ) for establishing level A IVIVC. Target times were computed to obtain guidelines for future product design by predicting FRT ᵥᵢₜᵣₒ by substituting FRT ᵥᵢᵥₒ in the IVIVC equation of the Test product.

List of Figures

FIG. 1 PULSATILE MACROPARTICULATE DRUG DELIVERY SYSTEM (Showing hypothetical cumulative in-vivo dissolution profiles)

FIG. 2 HYDROPHILIC MATRIX DRUG DELIVERY SYSTEMS (Polyexponential Fitting of Fractional Release Time Based IVIVC)

FIG. 3 HYDROPHILIC MATRIX DRUG DELIVERY SYSTEMS (Simulation of Claritin D—12 Hour.)

FIG. 4 HYDROPHILIC MATRIX DRUG DELIVERY SYSTEMS (Fractional Release Time Based IVIVC for Claritin D—24 Hour.)

FIG. 5 HYDROPHILIC MATRIX DRUG DELIVERY SYSTEMS (Predicted in-vivo cumulative amounts for 12 Hours based on FRT IVIVC for 24 Hours of FIG. 3.)

FIG. 6 HYDROPHILIC MATRIX DRUG DELIVERY SYSTEMS (Predicted Human response for 12 Hours based on convolution of in-vivo amounts in FIG. 4.)

FIG. 7 DELAYED RELEASE DRUG DELIVERY SYSTEM (Showing hypothetical cumulative in-vivo dissolution profiles)

FIG. 8 DELAYED RELEASE DRUG DELIVERY SYSTEM (Showing hypothetical cumulative in-vivo dissolution profiles)

FIG. 9 DIFFUSION CONTROLLED DRUG DELIVERY STSTEM. (Linear Fitting of Fractional Release Time Based IVIVC)

1. A fractional release time-based approach for correlating in-vitro to in-vivo data for targeting optimum therapeutic ranges, while reducing the criticality of discriminating in-vitro dissolution procedures, especially when conventional modeling methods are poorly applicable.

2. The fractional release time-based approach for correlating in-vitro to in-vivo data as defined in claim 1 wherein the drug molecules are acidic, basic. or amphoteric in nature.

3. The fractional release time-based approach for correlating in-vitro to in-vivo data as defined in claim 2 wherein the drug molecules are soluble or poorly soluble in the gastrointestinal media.

4. The fractional release time-based approach for correlating in-vitro to in-vivo data as defined in claim 3 wherein the molecules are high permeability lipophilic drugs.

5. The fractional release time-based approach for correlating in-vitro to in-vivo data as defined in claim 3 wherein the molecules are upper gastrointestinal high permeability hydrophilic drugs

6. The fractional release time-based approach for correlating in-vitro to in-vivo data as defined in claim 1 wherein the drug delivery system is extended release.

7. The fractional release time-based approach for correlating in-vitro to in-vivo data as defined in claim 1 wherein the drug delivery system is delayed release.

8. The fractional release time-based approach for correlating in-vitro to in-vivo data as defined in claim 1 wherein the drug delivery system is biphasic extended release.

9. The fractional release time-based approach for correlating in-vitro to in-vivo data as defined in claim 1 wherein the drug delivery system is pulsatile.

10. The fractional release time-based approach for correlating in-vitro to in-vivo data as defined in claim 1 wherein the drug delivery system is immediate release and delayed extended release.

11. The fractional release time-based approach for correlating in-vitro to in-vivo data as defined in claim 1 wherein the drug delivery system is extended release and delayed release.

12. The fractional release time-based approach for correlating in-vitro to in-vivo data as defined in claim 1 wherein the drug delivery system is upper gastrointestinal-specific.

13. The fractional release time-based approach for correlating in-vitro to in-vivo data as defined in claim 1 wherein the drug delivery system is colonic-specific.
